(19) United States
(12) Patent Application Publication
(10) Pub. No.: US 2006/0182748 A1
(43) Pub. Date: Aug. 17, 2006

(54) Method of creating antivenom using Emus

(76) Inventors: Maurine Pearson, Pilot Point, TX (US); Teresa L. Barr, Hood River, OR (US)

Correspondence Address: Peter A. Borsari, 3 South Fieldway, Rehoboth Beach, DE, 19971, US

(21) Appl. No.: 11/374,681

(22) Filed: Mar. 14, 2006

Related U.S. Application Data
(63) Continuation-in-part of application No. 10/245,940, filed on Sep. 18, 2002, now abandoned.

(51) Int. Cl. A61K 39/395; C12P 21/04; C07K 16/40

(52) U.S. Cl. 424/146.1; 435/70.21; 530/388.26

(57) ABSTRACT

A method for creating an antivenom for snake bites comprising obtaining the venom from at least one poisonous snake, obtaining an adult ratite bird, injecting the adult ratite bird with the venom in appropriate amounts at appropriate intervals such that the ratite bird develops antibodies to the venom, extracting blood from the bird or harvesting an egg from the inoculated ratite and removing the desired antibodies. The desired antibodies can be used to produce a antiserum containing antibodies to the venom.

### RELATED APPLICATION INFORMATION

This application is a continuation-in-part application of U.S. patent application Ser. No. 10/245,940, filed in the United States Patent & Trademark Office on 18 Aug. 2001 and claims the benefit of priority of therefrom.

### FIELD OF INVENTION

The present invention relates to methods of use for the treatment of venomous snake bites, poisonous spider bites and viral infections and compositions derived from such methods. More specifically, the present invention relates to a method of producing antivenom using Emus, particularly Emu eggs.

### BACKGROUND OF THE INVENTION

Venomous snakes are found throughout the world and cause more than three million snake bites a year worldwide (European Bioinformatics Institute). The venom from such snakes are composed of about 90% proteins and contain a number of active toxins that are used to paralyze and capture prey. The effects of these toxins include pro- and anti-blood coagulation, neurotoxicity, mycotoxicity, nephrotoxicity, cardiotoxicity and necrotoxicity. Of the several types of toxins found in venom, neurotoxins and hemotoxins have been studied extensively. Neurotoxins attack the victim's central nervous system and usually result in heart failure and/or breathing difficulties. Hemotoxins attack the circulatory system and muscle tissue causing excessive scarring, gangrene, permanent disuse of motor skills, and sometimes leads to amputation of the affected area.

Neurotoxins can be classified according to their site of action: pre-synaptic neurotoxins block neurotransmission by affecting acetylcholine transmitter release while post-synaptic neurotoxins are antagonists of the acetylcholine receptor. Together these neurotoxins effectively block skeletal neuromuscular transmission by crippling receptors, while at the same time acting to destroy any neurotransmitter that might compete with the toxin for receptor binding.

Antivenom was developed more than a century ago to counteract the effect of snake bites. The antivenom is produced by “hyper-immunizing” an animal against a snake bite by graduated and increased regular dosage of that animal with the venom of a snake and extracting antivenom serum from the immunized animal. The animal most often used to create antivenom is the horse. Venom is injected into the horse, slowly increasing the amount as the horse builds up antibodies to the venom. Blood is taken from the immunized horse and the serum containing the antibodies then is separated.

There has been little change in antivenom production techniques since its early development. Horses are inoculated with small amounts of venoms “milked” from the fangs of poisonous snakes, the horse's blood serum is collected and the antibodies to the snake venom proteins are harvested. When injected into the blood of a person who has suffered a snakebite, those antibodies bind to circulating venom proteins and neutralize them.

Today, there are two antivenom products available in the United States, both developed by Wyeth-Ayers Laboratories in Philadelphia, Pa. using the horse blood serum production technique. A significant drawback to the use of antivenom derived from horse serum is that such antivenom can cause serious hyper allergic reactions termed serum sickness. Common symptoms, sometimes referred to as serum sickness, include fever, rashes, nausea and muscle weakness, sometimes followed by nerve inflammation and permanent muscle atrophy. In some instances, the treated person can go into anaphylactic shock. These reactions are due to the extraneous horse-derived proteins found in the antivenom which the human body recognizes as foreign.

There has been some research into developing antivenom using sheep rather than horses. While sheep antibodies appear to cause fewer allergic reactions in people, serum sickness is still common.

More recently, there has been increasing interest in using chickens, and in particular, chicken eggs, to produce antivenom. The method comprises injecting venom into chickens, which produce antibodies that become concentrated in the yolks of the eggs of the chickens. The antivenom antibodies are separated from the egg yolks and purified to produce the antivenom. A typical procedure for purifying the antibodies by affinity purification, in which an antivenom antibody solution is passed through a column having venom proteins. Only venom-specific antibodies stick to the column, while extraneous proteins flow through. An advantage of using chicken eggs to produce antivenom is that although egg proteins can trigger allergic reactions in some people, the chicken protein does not cause the intense symptoms of serum sickness and anaphylaxis.

U.S. Pat. No. 5,196,193 to Carroll, issued Mar. 23, 1993, discloses the production of antivenoms in non-mammals and particularly by using chicken eggs. Carroll describes immunoaffinity purification as the preferred method of separating the antivenom antibodies. However, while some believe that the allergenic reaction is reduced by producing antivenom from chicken eggs, there still is considerable dispute as to whether more people are allergic to chickens than horses. In addition, there is another significant drawback to producing antivenom using chicken eggs and that is volume. It has been reported that the amount of antivenom antibody derived from a liter of horse blood is the equivalent of antivenom antibody produced by 50 chicken eggs. Another report suggests that the average yield of antivenom antibody is about 1.0 milligram per chicken egg. Since the average snakebite victim requires between 500 and 1,000 milligrams of antivenom, at least 40 dozen chicken eggs are required for one therapeutic dosage.

Consequently, a need still exists for a method to produce antivenom which is less likely to create allergic reactions and which can be economical to produce in large volumes. The present inventors have discovered that using Emu eggs to produce antivenom meets this need.

Found in the wild only in Australia, Emus are the second largest members of the ratite group of flightless birds in the world. The ratite family includes the Emu, ostrich, rhea, cassowary, and kiwi. The Emu have wings but they are very tiny. They can run up to 30 miles an hour, as they have very large and strong legs. Although a very docile creature, the Emu's legs are so strong that one kick can break a man's leg. Emus are by nature, very healthy, are immune to many diseases and are to parasites, such as ticks, fleas, and mites. This immunity makes these ratite birds a perfect choice for growing antigens. Emus are referred to as “living dinosaurs”, as their skeletal structure closely resembles some dinosaurs, and, in fact, Emus living today closely resemble their ancestors of millions of years ago. Emus now are being farmed in many parts of the world. They are raised for their valuable products, which include very low fat meat, supple leather hides, decorative and nutritional eggs, and very rich oil, hereinafter referred to as “Emu oil”.

Emu oil is obtained from the fat of the Emu. It is an all-natural substance. Emu oil contains high amounts of EFA's (essential fatty acids). EFA's produce energy in the process of oxidation. In humans, EFA's govern growth, vitality and mental state of mind. Oxidation is the central and most important living process in our body. In fact, the EFA structure of the Emu oil is very close to the structure of the human skin oil as shown in the following table.

Fatty Acid Composition of Emu Oil vs. Human Skin Oil

| Component | | Emu Oil | Human Skin Oil |
|---|---|---|---|
| Myristic | C:14:0 | 0.3% | 2.1% |
| Palmitic | C:16:0 | 20.3% | 20.2% |
| Palmitoleic | C:16:1 | 3.2% | 3.8% |
| Margaric | C:17:0 | 0.2% | |
| Margaric oleic | C:17:1 | 0.1% | |
| Stearic | C:18:0 | 10.1% | 11.2% |
| Oleic | C:18:1 | 51.6% | 30.8% |
| Linoleic | C:18:2 | 13.1% | 15.1% |
| Linolenic | C:18:3 | 0.5% | 0.3% |
| Arachidic | C:20:0 | 0.1% | |
| Eicosinoac | C:20:1 | 0.5% | |

Calculated iodine value 69.7-72.8 mEq/100 g
OSI - 11.95 Hours @ 110.0 degrees

Other fatty acids also found in Emu oil include elaidic acid and vaccenic acid.

An analysis of the fatty acids found in Emu oil reveals that the oil contains approximately 60-70% of fatty acids most of which are unsaturated fatty acids. The major fatty acid found in Emu oil is oleic acid, which is monosaturated and which comprises over 40% of the total fatty acid content of the Emu oil. Emu oil also contains two essential fatty acids which are important to human health: 20% linoleic acid and 1-2% alpha-linolenic acid.

Essential fatty acids (EFA's) play two important roles in human physiology, both of which are derived from their incorporation into the phospholipids of cell membranes. By virtue of their high degree of unsaturation, and hence low melting points, EFA's decrease membrane viscosity and affect several aspects of membrane function, including, but not limited to anti-inflammatory properties. An allergenic response is an over-blown inflammation response. Since Emu oil comprises from about 60 to about 70% fatty acids, it reduces inflammation in humans and enhances healing. Consequently, it is evidential that the Emu antigens are likely to be less allergenic than that of antigens grown in traditional antigen mediums.

Emus have been tested by Dr. Warren Burggren (with the University of North Texas system) and have been determined to have a cardiovascular system closely resembling that of humans. Dr. Burggren is quoted to say that “hearts in the eggs of Emus are very similar to human hearts in their early stages of development”, page 20, dated 2001, Resource, a periodical published by University of North Texas, Office of University Communications and Marketing published at 3500 Camp Bowie Blvd, Ft. Worth Tex., 76107-2699.

In looking at the profile of the similarities between human skin oil and Emu Oil, as well as the respective lipid profiles, and the similarity between the human heart and the Emu, it would be likely that a common thread exists between the two. Therefore, due to the fact that emus are disease resistant and have a low immunology profile, antibodies grown in emus create less allergenic reaction than antigens grown in traditional mediums. Traditional mediums include, for example, use of horses, chicken eggs, sheep, porcine (pig) pancreatic hydrolysate of casein, VERO cells; a continuous line of monkey kidney cells, or fetal rhesus, monkey lung cells, as well as yeast derivatives.

Since Emu oil is more compatible with the human body oil, it produces fewer allergic reactions. Many people cannot receive life saving vaccines because they are allergic to chicken or horse and their bodies cannot tolerate the presence of those components in the serum. Life saving vaccines include anti-venom serums as well as viral vaccines, including, but not limited to, influenza virus, mumps virus, measles virus, rubella virus, diphtheria virus, tetanus virus, small pox virus, tuberculosis, rotavirus, varicella, pertusis (whooping cough), HIB, pneumoccal, meningoccal, cholera, rabies virus and poliovirus.

One antivenom serum is snake antivenom. Not only are the compositions of Emu oil and human skin oil close in comparison, as is the hearts of Emus and humans, Dr. Warren Burggren also has discovered the same common thread and similarity between the cardiovascular systems of snake embryos and human heart embryos. This would further the argument for using the Emu, since it would appear that the Emu, the snake and the human all have a “common thread” according to Burggren.

Snakes propagate very badly and slowly in captivity. So, to obtain their venom, snakes often have to be caught in the wild, another lengthy, difficult and dangerous task. Besides this reality, the venom composition depends on the snake's age, sex, season and other factors. For all of these reasons, the ideal snake anti-venom production system would be one that didn't involve the reptiles at all to multiply the anti-venom. Emus are a natural choice to multiply anti-venom, as they are disease resistant and their cardiovascular system, as well as their skin oil, is similar humans.

A need has long existed for a method of producing antivenom in high volumes in a cost effective and economical manner. Such a method should not require the use of horses, and other traditional medium which can result in severe allergic reactions in humans. In addition, such a method should be capable of producing vaccines, particularly for small pox, diphtheria, measles, mumps, rubella, polio, tetanus, pertusis (whooping cough), HIB, pneumonia, meningitis, influenza, hepatitis A, tuberculosis, and cholera, that were not derived from chicken eggs. Moreover, such an invention should be capable of producing a vaccine for any existing live and killed pandemic viruses, including but not limited to, influenza virus, mumps virus, measles virus, rubella virus, diphtheria virus, tetanus virus, small pox virus, tuberculosis, rotavirus, varicella, pertusis (whooping cough), HIB, pneumoccal, meningoccal, cholera, rabies virus and poliovirus.

### SUMMARY OF THE INVENTION

Accordingly, it is an object of the present invention to provide a method for increasing the production of antivenom in a cost effective and economical manner.

It also is an object of the present invention to provide a method to grow a greater amount of vaccine at one time for harvest.

It is another object of the present invention to provide a method for producing antivenom that does not require the use of horses or other growing mediums that can cause allergic reactions.

It is still another object of the present invention to provide a method of creating an antivenom for snake bites in an adult ratite bird.

It is yet another object of the present invention to provide a method of creating an antivenom for spider bites in an adult ratite bird.

It is a further object of the present invention to provide a method of creating vaccines, particularly for small pox, diphtheria, measles, mumps, rubella, polio, tetanus, pertusis (whooping cough), HIB, pneumonia, meningitis, influenza, hepatitis A, tuberculosis, and cholera, in eggs of an adult ratite bird.

It is still a further object of the present invention to provide a method of creating a vaccine for any existing live and killed pandemic viruses, including but not limited to, influenza virus, mumps virus, measles virus, rubella virus, diphtheria virus, tetanus virus, small pox virus, tuberculosis, rotavirus, varicella, pertusis (whooping cough), HIB, pneumoccal, meningoccal, cholera, rabies virus and poliovirus, in eggs of an adult ratite bird.

These and other objects of the present invention are accomplished, in one embodiment, by a method for creating antivenom for snake bites in an adult ratite bird. This embodiment involves obtaining the venom of a poisonous snake, obtaining an adult ratite bird, injecting the bird with the snake venom in small amounts, and increasing the tolerance of the ratite bird to the venom. The venom injections are continued until the bird does not exhibit symptoms of the venom in the bird. The method then involves extracting blood from the bird, removing desired antibodies, and using the desired antibodies to make an antiserum containing antibodies to the venom.

Another embodiment of the present invention is a method for creating anti-venom for spider bites in an adult ratite bird. This method is similar to the method for creating anti-venom for snake bites, except that the venom of a spider is used in an adult ratite bird. The method then involves extracting blood from the bird, removing desired antibodies, and using the desired antibodies to make an antiserum containing antibodies to the venom.

Still another embodiment of the present invention is a method for making a vaccine using the egg of a ratite bird, by obtaining an embryonated egg of a ratite bird, obtaining seed viruses in a suitable carrier, inoculating the egg with the seed virus using a syringe, and, then, incubating the egg. The method involves separating the albumin from the yoke of the egg, removing the protein from the antibodies forming an antibody concentrate, and mixing the antibody concentrate with a carrier.

Additional objects, advantages and novel features of the invention will be set forth in part of the description which follows, and in part will become apparent to those skilled in the art upon examination of the following specification or may be learned by practice of the invention.

### DETAILED DESCRIPTION

The present invention relates to a method for producing an antivenom for counteracting the venom of venomous snake bites and poisonous spider bites. The present invention also relates to a method of creating a serum for combating the flu, any pandemic virus, and similar viruses. The present invention comprises a method of inoculating a ratite bird, in particular the Emu, with snake venom, spider venom or a specific virus to create an immunized Emu and then extracting antibodies from the immunized Emu.

In one embodiment, Emu eggs are utilized to increase production of snake antivenom. The use of Emu eggs has a number of advantages of conventional methods, particularly over the use of chicken eggs. As discussed previously, Emu oil is less likely to cause an allergenic reaction. Vaccines grown in Emu serum are less likely not to cause allergic reactions, such as tetanus shots, in humans due to the closeness of compatibility.

In addition, a far greater amount of anti-venom can be produced using Emu eggs rather than chicken eggs. Emu eggs have a volume of about 500 to about 850 ml and are equivalent to about 10 to 15 large chicken eggs. Without the shell, chicken eggs contain about 65% white, and 35% yolk. By comparison, Emu eggs contain 55% white and 44% yolk. This is one noticeable difference between chicken and Emu eggs. The Emu's incubation period is 56 days as versus the chicken at 21 days. The chicken egg contains approximately 43.0% cholesterol compared to 89.0% in Emu eggs. Emu's eggs also contain less water than chicken eggs, 67.0% compared to 75.0% in chicken eggs. The white of the Emu eggs contains mostly water, about 90%, with about 9% protein and no cholesterol. The yolk of the Emu contains 45-50.0% moisture, 15% protein, 1.5% cholesterol and 30-40.0% lipids. The high protein content of the yolk of the Emu egg makes the Emu egg the perfect choice for growing antigens for antibiotic production and vaccines, as antigens attach themselves to the protein of the embryonic fluid. Thus, Emu eggs are far more preferable than chicken eggs, due to their considerable size and potential for vaccine production.

Since Emu eggs are 10 to 15 times larger than chicken eggs and contain a higher percentage of yolk, it is believed that a single Emu egg could be capable of producing at least 10 to 15 times the amount of antivenom antibody produced the a single chicken eggs. This yield represents a significant increase over the amount yielded from a chicken egg.

Since it takes approximately fifteen chickens a day to lay fifteen eggs, this inventive method saves the filth produced by fifteen chickens, the food for fifteen chickens, the space of fifteen chickens, the need for chicken coops, the need for chicken medicines to keep the chickens healthy. In contrast, an Emu can lay nine eggs per month, the equivalent 135 chicken eggs per month. Unlike chickens, Emus are not fussy animals. Emus are weather tolerant and can get wet, stay outside in the cold under freezing temperatures (as long as they don't get frostbitten) and be outside in the open up 110° F., shade or no-shade. Emus eat bugs, grasses, and have no need for special food in order to lay eggs, although a Emu supplemental feed is favorable to ensure optimum health and overall wellness of the birds.

Overall, since fifteen chickens cost more to maintain and have a higher chance of dying than one Emu, and since the average Emu lives to be about 30 years old and the average chicken only lives to be about 7 years old, this inventive method is more economical by almost 50% than known techniques. Moreover, a typical Emu costs, at most about $65 per year to feed and maintain. A typical chicken costs about $20-25 per year. Clearly, using Emus is much less expensive than conventional methods using chicken eggs to grow anitvenoms and vaccines. Therefore, antibiotics and vaccines are better grown in Emu eggs versus chicken eggs because they can hold more volume and produce more antibody.

Moreover, since Emu embryos have a cardiovascular system that is similar to human cardiovascular systems and Emu oil is similar to human skin oil, antibiotics and vaccines are more compatible and reduce allergic reactions to vaccinations and the like.

In one embodiment of the present invention, a method for producing snake antivenom is provided. The method comprises obtaining the venom from at least one venomous snake, obtaining a ratite bird, in particular in Emu, inoculating or injecting the adult ratite bird with the venom in amounts capable to induce antibody formation to the venom, thereby causing the Emu to create immunity to the disease. Typically, the Emu is injected over a period of time with venom in small amounts ranging from about 5.0 mg of detoxified toxin to about 25.0 mg of detoxified toxin, thereby increasing the tolerance of the Emu to the venom. Preferably, the Emu is inoculated over a period of several weeks by increasing amounts of the venom. For example, the Emu is injected on a first day with a venom having 5.0 mg of detoxified toxin, is injected on another day with a venom having 10.0 mg of detoxified toxin, on still another day with venom having 15.0 mg of detoxified toxin, with continued venom injections up to 25.0 mg of detoxified toxin. The venom injections continue until the Emu does not exhibit symptoms of the venom and is immunized to the venom. A booster inoculation of venom containing about 5.0 mg of detoxified toxin can be given to the Emu once immunity has been established. In this manner, the Emu develops antivenom antibodies to the venom. The antivenom antibodies are found in the blood of the Emu. An automated laser flow-cytometric method may be used to tabulate and validate antibody levels in the ratite bloodstream. The immunized Emu then will lay eggs having antivenom antibodies. The antivenom antibodies can be extracted from the yolk of the Emu egg using standard purification methods which are well known to those skilled in the art. Suitable purification methods include T gel purification and affinity purification. Once the antivenom antibodies are removed, an antivenom serum can be produced. The yield of antivenom antibody from a single Emu egg is in the range of about 2.0 milligrams to about 20.0 milligrams, the yield dependent in part on the amount of antivenom antibodies the Emu has produced to the toxin inoculation process.

The method for the production of antivenom of snake bites also can comprise extracting one percent of blood of the immunized Emu, which can be collected via the jugular vein or wing, using a 3-10 ml syringe and a 20-22 gauge needle. The extracted blood is separated using conventional techniques such that a fluid of serum or antitoxin is separated from clotted blood. The fluid of serum containing antitoxins or antivenom antibodies is referred to as blood serum. The antivenom antibodies are recovered from the blood serum as described above.

The venom can be obtained from any venomous snake, suitable examples of which include coral snake, pit viper, copper head, cottonmouth, rattle snake, western Mississauga snake, water moccasin, western pigmy snake, western diamond back, cane brake snake, Mojave snake, mottled rock snake, banded rock snake, black tailed snake, prairie snake, and south Texas rattle snake.

In another embodiment, the present invention provides a method for creating antivenom for spider bites in an adult ratite bird, in particular an Emu. The method involves obtaining the venom from at least one poisonous spider, obtaining an adult ratite bird, injecting the adult ratite bird with the venom in small amounts as described above until the bird does not exhibit symptoms of the venom, and removing the desired antibodies either from the blood or eggs, and using antibodies and make an antiserum containing antibodies to venom. The method for creating anti-venom for spider bites can include extracting between 6.0% and 10.0% of the blood volume of the adult ratite every 2 to 3 weeks.

Examples of spiders for which antivenom can be produced by the method of the present invention include the brown recluse spider, black widow spider, brown spider, widow spider, red widow spider, and northern widow spider.

In another embodiment, the method of the present invention uses Emu eggs to grow vaccines for various types of virus, including small pox, any pandemic virus and others. The resultant vaccine causes fewer allergic reactions in users and is more compatible with human blood systems than known vaccines and anti-snake bite serums grown in chicken eggs.

A large number of viruses can be used to create vaccines by this method, including but not limited to influenza virus, mumps virus, measles virus, rubella virus, diphtheria virus, tetanus virus, small pox virus, tuberculosis pertusis (whooping cough), HIB, rotavirus, varicella, pneumoccal, meningoccal, cholera, rabies virus and poliovirus.

Because Emu eggs hold more volume and produce more antibody, using Emu eggs is particularly advantageous for creating influenza vaccines which are grown for up to four (4) months. Another problem with growing vaccines using animal cells that are not human-like is that during serial passage of the virus thru the animal cells, animal RNA and DNA can be transferred from one host to another and undetected animal viruses may slip past quality control testing procedures, as shown in 1955 thru 1961 with SV40 (simian virus #40), meaning the 40th virus found which has oncogenic or cancer causing properties.

The micro-organisms, or serum, either bacteria or viruses, thought to be causing certain infectious diseases and which the vaccine is supposed to prevent are whole-cell proteins or just the broken-cell protein envelopes, and are called antigens. Antigens are a substance, usually a protein, on the surface of a cell or bacterium that stimulates the production of an antibody. Chemical substances that are supposed to enhance the immune response to the vaccine, called adjuvants, can be injected along with the antigen to enhance the immune response stimulated by the antigen. An adjuvant is a drug or agent added to another drug or agent to enhance its medical effectiveness. Chemical substances which act as preservatives and tissue fixatives, which are supposed to halt any further chemical reactions and putrefaction, such as decomposition or multiplication of the live or attenuated or killed biological constituents of the vaccine, also can be injected along with the antigen.

Adjuvants, preservatives and tissue fixatives can be formaldehyde, thimerosal, aluminum hydroxides and aluminum phosphates, polysorbates 80 and 20, gelatin, hydrolyzed gelatin and processed gelatin, glycerol, sucrose, sorbitol, formalin, sodium chloride, phenoxyethanol, betapropiolactone, phenol red, monosodium glutamate, potassium monophosphates, tri (n) buytlphosphate, lactose, ammonium sulfate, residual MRC5 from the medium, and the like.

The desired antibodies which can be produced by the method of the present invention can be developed for influenza virus, mumps virus, measles virus, rubella virus, diphtheria virus, tetanus virus, small pox virus, tuberculosis, rotavirus, varicella, pertusis (whooping cough), HIB, pneumoccal, meningoccal, cholera, rabies virus, and poliovirus, as well as snake antivenom and spider antivenom.

The method of the present invention also contemplates producing and anti-serum from the antibodies produced by the immunized ratite bird. The anti-serum can comprise suitable adjuvants for injection, such as, adjuvants, tissue fixatives and preservatives, formaldehyde, thimerosal, aluminum hydroxides and aluminum phosphates, polysorbates 80 and 20, gelatin, hydrolyzed gelatin and processed gelatin, glycerol, sucrose, sorbitol, formalin, sodium chloride, phenoxyethanol, betapropiolactone, phenol red, monosodium glutamate, potassium monophosphates, tri (n) buytlphosphate, lactose, ammonium sulfate, residual MRC5 from the medium, and the like. The injections in the method can be in the amount of 1-3 ml per day.

The present invention also contemplates a method for making a vaccine using the egg of a ratite bird. The method involves obtaining an adult ratite bird, obtaining seed viruses in a suitable carrier, inoculating the egg adult ratite with the seed virus using a syringe, obtaining an egg from the inoculated bird separating the albumin from the yoke of the egg, removing the protein from the antibodies forming an antibody concentrate, and mixing the antibody concentrate with a carrier, adjuvants, tissue fixatives and preservatives.

The seed viruses used in the method for making a vaccine using the egg of a ratite bird can be a member of the following group: live and killed pandemic viruses, which include, influenza virus, mumps virus, measles virus, rubella virus, diphtheria virus, tetanus virus, small pox virus, rabies virus and polio, tuberculosis pertusis (whooping cough), HIB, rotavirus, varicella, pneumoccal, meningoccal, cholera, rabies virus and poliovirus.

The incubation period in the method for making a vaccine using the egg of a ratite bird can be one week when the seed virus is a stockpiled pandemic virus. The protein in the method can be removed from the albumin by extraction. The carrier in the method can be adjuvants, tissue fixatives and preservatives, formaldehyde, thimerosal, aluminum hydroxides and aluminum phosphates, polysorbates 80 and 20, gelatin, hydrolyzed gelatin and processed gelatin, glycerol, sucrose, sorbitol, formalin, sodium chloride, phenoxyethanol, betapropiolactone, phenol red, monosodium glutamate, potassium monophosphates, tri (n) buytlphosphate, lactose, ammonium sulfate, residual MRC5 from the medium, and the like. The present invention also contemplates producing a vaccine for a member of the group of live and killed pandemic virus, influenza virus, mumps virus, measles virus, rubella virus, diphtheria virus, tetanus virus, small pox virus, rabies virus and poliovirus, tuberculosis pertusis (whooping cough), HIB, rotavirus, varicella, pneumoccal, meningoccal, cholera, rabies virus and poliovirus.

While particular embodiments of the invention have been described, it will be understood, of course, that the invention is not limited thereto, and that many obvious modifications and variations can be made, and that such modifications and variations are intended to fall within the scope of the appended claims.

What is claimed is:

1. A method of producing an antivenom for snake bites comprising
a. obtaining the venom of at least one poisonous snake;
b. obtaining an adult ratite bird;
c. inoculating the adult ratite bird with the venom of at least one poisonous snake in an amount capable of inducing antibody formation to the venom, in order to produce antivenom antibodies in an immunized adult ratite bird;
d. harvesting an egg from the inoculated adult ratite bird, said egg having a yolk containing antivenom antibodies;
e. extracting between about 2.0 milligrams and about 20.0 milligrams of antivenom antibodies from the yolk of the harvested egg, and
f. using the antivenom antibodies to produce an antivenom for snake bites.

2. The method of producing an antivenom for snake bites in accordance with claim 1, wherein said adult ratite bird is an Emu.

3. The method of producing an antivenom for snake bites in accordance with claim 1, wherein said adult ratite bird is inoculated by injecting the adult ratite bird with venom containing from about 5.0 mg of detoxified toxin to about 20.0 mg detoxified toxin over a period of time until the adult ratite bird has become immunized to the venom.

4. The method of producing an antivenom for snake bites in accordance with claim 3, wherein said adult ratite bird is inoculated by serially injecting the adult ratite bird with venom containing increasing amounts of detoxified toxin.

5. The method of producing an antivenom for snake bites in accordance with claim 1, wherein the immunized adult ratite bird is inoculated with a booster injection containing about 5.0 mg of detoxified toxin.

6. The method of producing an antivenom for snake bites in accordance with claim 1, wherein between about 2.0 milligrams to about 10.0 milligrams of antivenom antibodies are extracted from the harvested egg.

7. The method of producing an antivenom for snake bites in accordance with claim 1, wherein about 5.0 milligrams to about 10. milligrams of antivenom antibodies are extracted from the harvested egg.

8. The method of producing an antivenom for snake bites in accordance with claim 1, wherein the antivenom antibodies are extracted from the harvested egg using affinity purification.

9. The method of producing an antivenom for snake bites in accordance with claim 1, wherein the antivenom antibodies are extracted from the harvested egg using T-gel purification.

10. The method of producing an antivenom for snake bites in accordance with claim 1, wherein the venom is derived from at least one poisonous snake selected from the group consisting of coral snake, pit viper, copper head, cottonmouth, rattle snake, western Mississauga snake, water moccasin, western pigmy snake, western diamond back, cane brake snake, Mojave snake, mottled rock snake, banded rock snake, black tailed snake, prairie snake, and south Texas rattle snake.

11. A method of producing an antivenom for snake bites comprising
a. obtaining the venom of at least one poisonous snake;
b. obtaining an Emu;
c. inoculating the Emu with the venom of at least one poisonous snake in an amount capable of inducing antibody formation to the venom, in order to produce antivenom antibodies in an immunized Emu;
d. harvesting an egg from the immunized Emu, said egg having a yolk containing antivenom antibodies;
e. extracting between about 2.0 milligrams and about 20.0 milligrams of antivenom antibodies from the yolk of the harvested egg, and
f. using the antivenom antibodies to produce an antivenom for snake bites.

12. The method of producing an antivenom for snake bites in accordance with claim 11, wherein said Emu is inoculated by injecting the Emu with venom containing from about 5.0 mg of detoxified toxin to about 20.0 mg detoxified toxin over a period of time until the Emu has become immunized to the venom.

13. The method of producing an antivenom for snake bites in accordance with claim 12, wherein said Emu is inoculated by serially injecting the adult ratite bird with venom containing increasing amounts of detoxified toxin.

14. The method of producing an antivenom for snake bites in accordance with claim 11, wherein the immunized Emu is inoculated with a booster injection containing about 5.0 mg of detoxified toxin.

15. The method of producing an antivenom for snake bites in accordance with claim 11, wherein between about 2.0 milligrams to about 10.0 milligrams of antivenom antibodies are extracted from the harvested egg.

16. The method of producing an antivenom for snake bites in accordance with claim 11, wherein about 5.0 milligrams to about 10. milligrams of antivenom antibodies are extracted from the harvested egg.

17. The method of producing an antivenom for snake bites in accordance with claim 11, wherein the antivenom antibodies are extracted from the harvested egg using affinity purification.

18. The method of producing an antivenom for snake bites in accordance with claim 11, wherein the antivenom antibodies are extracted from the harvested egg using T-gel purification.

19. The method of producing an antivenom for snake bites in accordance with claim 11, wherein the venom is derived from at least one poisonous snake selected from the group consisting of coral snake, pit viper, copper head, cottonmouth, rattle snake, western Mississauga snake, water moccasin, western pigmy snake, western diamond back, cane brake snake, Mojave snake, mottled rock snake, banded rock snake, black tailed snake, prairie snake, and south Texas rattle snake.
